(19) United States
(12) Patent Application Publication
(10) Pub. No.: US 2008/0096978 A1
(43) Pub. Date: Apr. 24, 2008

(54) OPHTHALMIC SOLUTIONS

(76) Inventor: Fu-Pao Tsao, Lawrenceville, GA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/961,034

(22) Filed: Dec. 20, 2007

Related U.S. Application Data
(63) Continuation of application No. 11/078,209, filed on Mar. 11, 2005.
(60) Provisional application No. 60/553,880, filed on Mar. 17, 2004.

(51) Int. Cl. A61K 47/26

(52) U.S. Cl. 514/781

(57) ABSTRACT

An ophthalmic solution comprising a hydrogen peroxide source in an amount sufficient to result in between about 0.001% and about 0.01% by weight stabilized hydrogen peroxide in said formulation as a preservative, one or more ocularly-compatible hydrogen peroxide stabilizers, hydroxypropylmethylcellulose and sodium carboxymethylcellulose, as well as pharmaceutical compositions comprising such solutions, are provided.

The present invention relates to ophthalmic eye wetting or lubricating solutions that contain both hydroxypropylmethylcellulose (HPMC) and sodium carboxymethylcellulose (CMC), and pharmaceutical compositions that comprise such eye wetting or lubricating solutions. U.S. Pat. Nos. 5,725,887 and 5,607,698, which are both expressly incorporated by reference herein in their entirety, disclose and claim methods for the preservation of ophthalmic solutions using stabilized hydrogen peroxide and compositions so preserved. It has now been unexpectedly discovered that the comfort provided by HPMC-containing solutions can be enhanced when CMC is also included in the solution. It has also been surprisingly discovered that a synergistic increase in viscosity occurs in ophthalmic solutions that contain both HPMC and CMC.

Trace amounts of peroxy compounds stabilized with a hydrogen peroxide stabilizer, especially diethylene triamine penta(methylene phosphonic acid) or 1-hydroxyethylidene-1,1-diphosphonic acid may be utilized as a preservative for the eye wetting solutions or eye lubricating solutions of the invention. A hydrogen peroxide source is any peroxy compound that is hydrolyzed in water to produce hydrogen peroxide. Examples of hydrogen peroxide sources, which provide an effective resultant amount of hydrogen peroxide, include sodium perborate decahydrate, sodium peroxide and urea peroxide. It has been found that peracetic acid, an organic peroxy compound, cannot be stabilized utilizing the present system.

The ophthalmic demulcents that are contained in the compositions of the present invention are HPMC and CMC. The HPMC (E4M) used can have a viscosity of 2,765 cps for a 2% solution using Brookfield model DV-II+ Viscometer, with #52 spindle, at 1.5 rpm and 25° C. to measure viscosity. The CMC (Akucell AF 2781) used can have a viscosity of 15,570 cps for a 2% solution using Brookfield model DV-II+ Viscometer, with #52 spindle, at 1.5 rpm and 25° C. to measure viscosity. Suitable grades of HPMC are Methocel A, E, F, J, and K brand products from Dow Chemical and suitable grades of CMC are Akucell AF 2781, Aqualon 7H3SXF PH, 7L, 7M from Akzo Nobel, Aqualon. The HPMC concentration in the formulations of the invention can be between about 0.005% and about 1.0%, and the CMC concentration in the formulations of the invention can be between about 0.005% and about 1.0%. In one embodiment, the HPMC and CMC concentrations in a formulation of the present invention are about 0.3% and about 0.25%, respectively. Other demulcents that can be utilized in addition to HPMC and CMC are hydroxyethyl cellulose, hydroxypropyl methylcellulose and methylcellulose; dextran 70; gelatine; and polyols, such as glycerine, polyethylene glycol 300, polyethylene glycol 400, polysorbate 80, propylene glycol, polyvinyl alcohol and povidone.

Excipients of various types compatible with the present invention include, but are not limited to, polysorbate gelatin (Tween), dextrans, lanolin inositol phosphates, alkylsulfosuccinates, sulfosuccinamates, alkyl silicone sulfosuccinates, alkylpolyether carboxylates, alkylaryl polyethoxylamines, alkylarylsulfonates, alpha olefin sulfonates, alkyl sulfates, alkyl ether sulfates, alkanol amides and alkamides, alkylamphoterics, amphoterics based on alkyl imidazoline, betaines, alkylaminopropionates, alkyliminodipropionates, alkylamphoglycinates, alkylamphocarboxyglycinates, alkylamphocarboxypropinates, alkylamphopropionates, alkylamidopropylhydroxysultaines, alkyletherhydroxypropylsultaines, alkylamphopropylsulfonate, quaternary ammonium polymers, quaternary ammonium halides, polyacrylamide, polyacrylates, polyvinyl pyrrolidone, polyvinyl alcohol, alkylalcohol ethoxylates, hydroxyalkylcelluloses, alkylamidopropyl PG-dimonium chloride phosphates, alkylampho PG-glycinate phosphates, gyceryl monoalkylates, sorbitan alkylates (Spans), Pluronics, Tetronics, sodium alkyl sulfates, sodium butoxyethoxy acetate, phosphate esters, glycosides, polyglycosides, mannitol, sorbitol, polyoxyethylene alkyl ethers, grillosan, guar gum, sodium hyaluronate, polyoxyl 40 stearate and polyoxyolkylene dimethylpolysiloxane.

A hydrogen peroxide stabilizer, as used herein, means any of the known stabilizers of peroxy compounds including phosphonates, phosphates, stannates, etc. Physiologically compatible salts of phosphonic acids may also be used, such as diethylene triamine penta(methylene-phosphonic acid and physiologically compatible salts thereof and 1-hydroxyethylene-1,1,-diphosphonic acid and physiologically acceptable salts thereof. Other stabilizers of peroxy compounds useful in the practice of the present invention are disclosed in U.S. Pat. No. 5,725,887 at, inter alia, column 5, line 55 to column 6, line 34.

The above stabilizers can be used in almost all indications previously mentioned to which the invention is applicable. However, when the solution is to come in contact with a hydrogel soft contact lens, stannate stabilizers are to be avoided as they tend to “cloud” the lens material.

When the peroxy stabilizer is diethylene triamine penta(methylene-phosphonic acid, it can be present in the solution in an amount between about 0.001% and about 0.02% by weight of the solution, or in an amount between about 0.002% and about 0.012% by weight of the solution.

When the peroxy stabilizer is 1-hydroxyethylene-1,1,-diphosphonic acid it can be present in the solution in an amount between about 0.002% and about 0.2% by weight of the solution.

Stabilizers other than diethylene triamine penta(methylene-phosphonic acid and physiologically compatible salts thereof and 1-hydroxyethylene-1,1,-diphosphonic acid and physiologically acceptable salts thereof are employed in physiologically tolerable amounts.

Soluble alkaline earth metal salts can be used in the compositions and methods of the present invention in amounts between about 0.002% and 0.2% by weight of the preserved solution, or between about 0.01% and 0.1% by weight of the preserved solution. Water soluble salts of magnesium and calcium are such alkaline earth metal salts. Preserved solutions comprising about 0.01% and 0.1% alkaline earth metal salts are disclosed herein. The present inventor has discovered that addition of such soluble alkaline earth metal salts increases antifungal preservative efficacy in ophthalmic solutions preserved with low amounts of hydrogen peroxide.

The pH of the stabilized solution is between about 5.5 and about 8. Preferably, the pH of a stabilized hydrogen peroxide solution is between about 6.0 and 8.0, most preferable between about 6.5 and 7.5. The pH can be adjusted as desired by incorporation of suitable amounts of acid or base of a physiologically tolerable nature in the amounts employed, e.g., hydrochloric acid and sodium hydroxide.

There may be present in the preserved solutions according to the present invention one or more conventional, substantially inert, physiologically acceptable tonicity enhancing agents. Suitable such agents include, e.g., mannitol; sorbitol; glycerol; alkali metal halides; phosphates; hydrogen phosphate; and borates, such as sodium chloride, sodium phosphate monobasic and sodium phosphate dibasic; and polyols. The function of such tonicity enhancing agents is to assure approximate physiologic tonicity to the solution which is instilled in the eye or to help assure such tonicity upon dilution if dilution is necessary prior to contact with the eye due to peroxide content as indicated above.

Preferably sufficient tonicity enhancing agents are present in the solution so that it is substantially isotonic or, such that, upon decomposition or dilution of the hydrogen peroxide therein, the resulting solution is substantially isotonic, e.g., substantially equivalent in tonicity to a 0.9% by weight aqueous sodium chloride solution.

The solutions of the present invention ca n also include buffering agents such borate and phosphate buffers.

In general, the stabilized hydrogen peroxide solutions of the present invention are characterized by their extraordinary stability, even under accelerated conditions, e.g., by heating the solutions to 100° C. for 24 hours. Thus, the shelf-life of these compositions is enhanced. Moreover, the instant compositions are characterized by either physiological tolerability subsequent to hydrogen peroxide decomposition.

Another advantage in using hydrogen peroxide in ophthalmic solution s is that the trace amount of hydrogen peroxide, especially less than 100 ppm, is destroyed once the hydrogen peroxide comes in contact with the eye. For example, catalase existing in the eye tissue will cause the breakdown of the hydrogen peroxide into water and oxygen. As a result, the solution, upon application, becomes preservative-free and greatly minimizes adverse reactions. The problems associated with other preservatives, such as the inability to break down innocuous compounds, are eliminated.

Formulation of the solutions of the invention can be made in a ny conventional manner. For example, all of the components other than the hydrogen peroxide and water can be placed in a container and fresh, preferably concentrated, hydrogen peroxide added thereto with mixing. Alternatively, the dry components can be rubbed-up with a small portion of liquid stabilizer, then the remainder of the stabilizer added, followed by the hydrogen peroxide, and most of the water. The viscosity enhancing agent, i.e., thickener, can then be added or the formed solution can be added to the thickener. One of ordinary skill in the art will be aware of numerous variations in the manner of formulating the solutions of the invention.

The solutions of the invention can be packaged in any pharmaceutically acceptable packaging, but it is desirable to package them in squeezable plastic multi-dose containers, such as dropper bottles. Such bottles can be made, e.g., of polyethylene or polypropylene or mixtures thereof. A dropper bottle will typically dispense between about 25 mL per drop and about 50 mL per drop. Typically, between 1 drop and 10 drops, or between 1 drop and 5 drops, or between 1 drop and 3 drops are administered at one time when employing the solutions of the invention when wetting or lubricating an eye.

When it is desirable to “neutralize” the peroxide activity, any means known, such as rinsing, contacting the solution with platinum, catalase or any other substance known to decompose hydrogen peroxide, will suffice. Additional physiological compatible peroxide neutralizing agents include reducing agent, such as pyruvic acid and suitable salts thereof, such as the sodium salt.

The following examples are presented for illustrative purposes and are not intended to limit the scope of this invention, but to demonstrate the stability of the peroxy solutions as stabilized in accordance with the present invention. All parts are by weight unless otherwise indicated.

### EXAMPLES

TABLE 1

| | | | | Viscosity (cps), 1.5 RPM, |
|---|---|---|---|---|
| | | | | Spindle # 42, Temp. = 25° C., |
| Formulation | | % | % Na- | Brookfield viscometer model |
| No. | pH | HPMC | CMC | DVII+ |
| 1 | 6.874 | 0.3 | 0.5 | 244 |
| 2 | 6.885 | 0.3 | 0.4 | 154 |
| 3 | 6.790 | 0.3 | 0.3 | 68 |
| 4 | 6.980 | — | 0.9 | 242.0 |
| 5 | 6.986 | — | 0.54 | 50.0 |
| 6 | 6.984 | — | 0.414 | 26.0 |
| 7 | 6.981 | — | 0.234 | 8.0 |
| 8 | 6.982 | — | 0.097 | 2.0 |
| 9 | 6.973 | 0.9 | — | 132 |
| 10 | 6.977 | 0.54 | — | 36 |
| 11 | 6.977 | 0.36 | — | 8.0 |
| 12 | 6.983 | 0.18 | — | 2.0 |
| 13 | 6.999 | 0.09 | — | 0-2 |
| 14 | 6.997 | 0.18 | 0.18 | 16 |
| 15 | 7.011 | 0.155 | 0.53 | 100 |
| 16 | 6.977 | 0.45 | 0.45 | 210 |
| 17 | 6.953 | 0.09 | 0.09 | 6.0 |
| 18 | 6.980 | 0.09 | 0.81 | 232 |
| 19 | 6.951 | 0.72 | 0.18 | 160 |
| 20 | 6.949 | 0.54 | 0.16 | 66 |
| 21 | 6.989 | 0.18 | 0.72 | 232 |
| 22 | 6.981 | 0.81 | 0.09 | 166 |

Example 1 contains the indicated concentrations of HPMC and CMC in an aqueous solution with 0.32% sodium chloride, 0.2% boric acid, 0.12% KCl, 0.05% calcium chloride dihydrate, 0.01% magnesium chloride hexahydrate, 60 ppm Dequest® 2060 and 0.028% sodium perborate tetrahydrate.

Example 2 contains the indicated concentrations of HPMC and CMC in an aqueous solution with 0.32% sodium chloride, 0.3% boric acid, 0.12% KCl, 0.005% calcium chloride dihydrate, 0.01% magnesium chloride hexahydrate, 60 ppm Dequest® 2060 and 0.028%sodium perborate tetrahydrate.

Example 3 contains the indicated concentrations of HPMC and CMC in an aqueous solution with 0.4% boric acid, 0.35% NaCl, 0.12% KCl, 0.05% calcium chloride dihydrate, 0.01% magnesium chloride hexahydrate, 60 ppm Dequest® 2060 and 0.028% sodium perborate tetrahydrate.

Examples 4-19 and 20-22 contain the indicated concentrations of HPMC and CMC in an aqueous solution with 0.26% sodium chloride, 0.05% calcium chloride dihydrate, 0.01% magnesium chloride hexahydrate, 0.5% boric acid, 0.12% potassium chloride, 0.0024% citric acid monohydrate, 60 ppm Dequest® 2060 and 0.028% sodium perborate tetrahydrate.

TABLE 2

| | | Viscosity | Separate Formulations | | Sum of Viscosity of |
|---|---|---|---|---|---|
| Formulation | Conc. CMC, | (synergistic effect) | with Similar Amounts | | Separate Formulations |
| No. | HPMC (%) | [cps] | of CMC and HPMC | | [cps] |
| 1 | 0.3, 0.3 | 68 | 6 | (0.4% CMC) | 34 |
| | | | 11 | (0.36% HPMC) | |
| 16 | 0.45, 0.45 | 210 | 5 | (0.54% CMC) | 86 |
| | | | 10 | (0.54% HMPC) | |
| 17 | 0.09, 0.09 | 6 | 8 | (0.097% CMC) | 4 |
| | | | 13 | (0.09% HPMC) | |
| 20 | 0.16, 0.54 | 66 | 7 | (0.234% CMC) | 44 |
| | | | 10 | (0.54% HPMC) | |
| 15 | 0.53, 0.155, | 100 | 5 | (0.54% CMC) | 52 |
| | | | 12 | (0.18% HPMC) | |
| 1 | 0.5, 0.3 | 244 | 5 | (0.54% CMC) | 58 |
| | | | 11 | (0.36% HPMC) | |
| 14 | 0.18, 0.18 | 16 | 7 | (0.234% CMC) | 10 |
| | | | 12 | (0.18% HPMC) | |

Table 2, above, demonstrates the synergism with respect to viscosity that can be achieved in ophthalmic artificial tear formulations that contain both HPMC and CMC. In all cases, as demonstrated by Table 2, a formulation with a similar, but lower, total concentration of HPMC and CMC had a higher viscosity than the added viscosities of two solutions which individually contained either HPMC or CMC. So, Example 16, which contains 0.45% HPMC and 0.45% CMC, has a viscosity of 210 cps, while a solution of 0.54% CMC has a viscosity of 50 cps and a solution of 0.54% HPMC has a viscosity of 36 cps, giving a total additive viscosity of 86 cps. This surprising synergism in viscosity is found for all solutions tested that contain both CMC and HP MC.

### Example 23

A solution of 0.3% HPMC (grade E4M), 0.5% boric acid, 0.26% NaCl, 0.12% KCl, 0.3% CMC (Aqualon, 7H3SXF, PH), 0.05% calcium chloride dihydrate, 0.01% magnesium chloride hexahydrate, 0.0024% citric acid monohydrate, 0.0060% Dequest® 2060, 0.028% sodium perborate is prepared. The pH is adjusted to 6.934.

### Comparative Example 23

### Allergan's Refresh® Liquigel™ Containing 1% CMC

Each of seven subjects dropped 1-2 drops of the formulation of Example 23 and Comparative Example 23 into each one eye and waited for 2-5 minutes. The dropper bottles containing both formulations were unmarked. All of the subjects evaluated which formulation they preferred as providing the most comfortable feeling in the eye. Five out of the seven subjects favored the formulation of Example 23, and two favored the formulation of Comparative Example 23.

What is claimed is:

1. An ophthalmic solution comprising:
(a) a hydrogen peroxide source in an amount sufficient to result in between about 0.001% and about 0.01% by weight stabilized hydrogen peroxide in said formulation as a preservative;
(b) one or more ocularly compatible hydrogen peroxide stabilizers;
(c) hydroxypropylmethylcellulose; and
(d) sodium carboxymethylcellulose.

2. The solution of claim 1, wherein the concentration of hydroxypropylmethylcellulose is between about 0.005% and about 1.0%, and wherein the concentration of sodium carboxylmethylcellulose is between about 0.005% and about 1.0%.

3. The solution of claim 2, wherein the concentration of hydroxypropylmethylcellulose is about 0.3% and the concentration of sodium carboxylmethylcellulose is about 0.25%.

4. The solution of claim 3, wherein said hydrogen peroxide source is selected from the group consisting of hydrogen peroxide, sodium perborate, sodium peroxide and urea peroxide.

5. The solution of claim 4, wherein said one or more hydrogen peroxide stabilizers is selected from the group consisting of diethylene triamine penta(methylene phosphonic acid), 1-hydroxyethylidene-1,1-diphosphonic acid, and physiologically compatible salts thereof.

6. The solution of claim 5, wherein said stabilizer is 1-hydroxyethylidene-1,1-diphosphonic acid.

7. The solution of claim 5, wherein said stabilizer is diethylenetriamine penta(methylene phosphonic acid).

8. The solution of claim 6, wherein said solution comprises between about 0.002% and about 0.2% by weight 1-hydroxyethylidene-1,1-diphosphonic acid or physiologically compatible salt thereof.

9. The solution of claim 7, wherein said solution comprises between about 0.002% and about 0.012% by weight diethylene triamine penta(methylene phosphonic acid) or a physiologically compatible salt thereof.

10. The solution of claim 3, wherein said solution comprises about 0.01 to 1% hydroxypropylmethylcellulose and about 0.01-1% sodium carboxymethylcellulose.

11. The aqueous solution of claim 1, further comprising a tonicity adjusting agent and a buffering agent.

12. A pharmaceutical composition comprising a dropper bottle in which is disposed an ophthalmic solution according to claim 1.

13. A pharmaceutical composition according to claim 12, wherein said dropper bottle comprises polypropylene.

14. A pharmaceutical composition according to claim 12, wherein said dropper bottle comprises polyethylene.
